(19) United States
(12) Patent Application Publication
(10) Pub. No.: US 2007/0100003 A1
(43) Pub. Date: May 3, 2007

(54) Hybrid system for Gasification of Biomass and conversion to synthesis gas suitable for fuel synthesis, with 3 potential applications

(76) Inventors: James Leigh Holley, Muenster, TX (US); Aubrey Leon Harris, Muenster, TX (US)

Correspondence Address: James Leigh Holley, PO BOX 339, Muenster, TX, 76252, US

(21) Appl. No.: 11/163,730

(22) Filed: Oct. 28, 2005

(51) Int. Cl. C07C 27/06

(52) U.S. Cl. 518/702; 518/714; 518/703

(57) ABSTRACT

Technical challenges of biomass-to-fuels conversion prompted the development of this hybrid system for biomass gasification. In this device, the matter is first pyrolyzed and the resulting vapors are drawn off and run through the char and tar in the second stage with the process steam in a supercritical steam gasification. The resulting gases are then purified by use of an amine wash scrubber. An adiabatic pre-reformer is then employed to break down aromatic compounds that most likely exist in the gas even after steam gasification. This gas is then fed to the main steam reformer, and afterwards the gas is cooled to suitable reaction temperatures for fuel synthesis. With a ratio H/C of 2.0, the gas is suitable for Fischer-Tropsch fuel synthesis, methanol synthesis, or production of hydrogen and carbon dioxide by a water-gas shift.

### DESCRIPTION OF THE RELATED ART

Pyrolysis, or destructive distillation, has been used since ancient times. The Egyptians heated wood in the absence of air to produce tars and oils for their elaborate funerary practices, and shipbuilders of the Middle Ages used it to make tars from pine to caulk seams and reinforce rope. This pitch was also used in the manufacture of torches, and mainly derived from pine wood which is well-known for its tarry characteristics and resultant flammability. The Industrial Age brought the use of pyrolysis to manufacture chemicals such as acetone, acetic acid, and especially methanol. From this comes the name “wood alcohol” for methanol.

In fact, methanol was first isolated by Robert Boyle, who in The Sceptical Chymist describes isolating it by the pyrolysis of boxwood: he thus named it “spirit of box”. Later works on pyrolysis of other woods described a mixture of liquids known as “pyroligneous acid” that was a mixture of various liquids, including furfural, acetone, methanol, and acetic acid. Methanol itself became known as “pyroxylic spirit”. In 1834, the French chemists Jean-Baptiste Dumas and Eugene Peligot determined methanol's elemental composition, and gave it the name methylene, from the Greek methu, meaning wine, and hyle, meaning wood. This name was shortened to methanol in 1892.

Methanol in and of itself can also be used as a fuel. It is regulation in most forms of open-wheel racing, and in Top Fuel racing nitromethane, a derivative of methanol and nitric acid, is the only allowable fuel. Unfortunately, its usefulness for ordinary consumers is limited. Methanol is much harsher to a gasoline engine than gasoline, and using it would require special modification that most people are either unable to afford or unwilling to have done to their vehicles. Methanol also, of course, does not run in a diesel engine. It can, however, be turned into fuel by means of a catalytic process using an acidic zeolite originally invented by Mobil in the mid-1970's. This fuel, unfortunately, suffers from a high proportion of aromatic compounds and violates many emissions standards; it is also prone to form aromatic solids upon standing. Despite this, a methanol-to-gasoline plant was built by Mobil in New Zealand in the late 1970's—early 1980's. It was sold to Methanex when oil prices fell again and production became uneconomical, and ran until 1997.

The rise of fossil fuel meant the demise of the pyrolysis plants, and the last one in America closed down in the 1950's. Modern methanol synthesis comes from the production of methane-rich natural gas. First, the methane is reformed with steam under a nickel-bearing catalyst to produce a synthesis gas. This can also be accomplished by blowing steam through hot coke. The result of methane steam reforming has a roughly 3:1 molar ratio of hydrogen to carbon monoxide, and if a 2:1 ratio is more desirable (such as in Fischer-Tropsch synthetic oil processes and methanol formation) a water-gas shift reaction is performed to adjust the hydrogen level. Then in the next reactor, carbon monoxide and two molar equivalents of hydrogen are combined using a copper-containing catalyst to produce methanol. It is also used to make formaldehyde, to denature ethanol, and as a solvent.

Recently, there has been a renewed interest in pyrolysis and other methods of converting biomass to fuel. One of the primary methods known to the art is partial oxidation, in which the material is burnt using only about ⅓ of the oxygen required for full combustion. Unfortunately, this process so far has only resulted in a medium-value heating gas, and requires an air liquefaction plant, a humongous consumer of energy, to be done properly. If done with atmospheric air, the resulting gas has very high nitrogen content. If it were suitable for synthesis, it would be better off used to produce ammonia. It also is not suitable for synthesis due to a low hydrogen and high carbon dioxide content, along with high tar contents which mean near instant death for any catalyst involved due to severe coking.

Steam gasification, so far, has been the more promising of the two approaches, and is a very old approach. In fact, steam blasted through hot coke was the first method of producing synthesis gas for ammonia and synthetic methanol production. This has had mixed results when applied to biomass, however. The high oxygen content of biomass ensures a high carbon dioxide level in the resulting gas, and the reaction itself is endothermic much like steam-methane reforming is. Additionally, water must be heated, which is an additional energy user due to the high specific heat of water. Nevertheless, steam gasification has been the preferred industrial process for years (mainly on coal) simply by virtue of the superior hydrogen content of the resulting gas.

It is the object of the present invention to overcome the technical challenges associated with biomass gasification, including content of tar and trace minerals, especially sulfur; to improve the hydrogen content of said gases; and to reduce the carbon dioxide content to an allowable level.

The device, in its preferred embodiment, is a continuous process, in which the carbon material involved is ground into slurry and conveyed by means of Archimedean screw through a section of pipe which is heated to 500 degrees Celsius with the use of electrical elements, which begins the process of pyrolysis. Pyrolysis vapors are drawn off in the first vessel and instantly routed to the second vessel along with the process steam, which serves both as compression agent and gasifier. The solids and the vapors both are reacted, the vapors and steam through the solids at the bottom of the reactor, at 800 degrees Celsius and no less than 60 atmospheres, wherein the formation of CO and hydrogen are both promoted and all carbon material converted.

This raw gas, consisting of mainly methane, CO, and hydrogen with less than 5% carbon dioxide, hydrogen sulfide, and ammonia by mole, is cooled to 25 degrees Celsius to prepare it for entry into the amine wash scrubber unit, in which it is passed through a solution containing no less than 50% ethanamine and 50% water for purification of the gases, removing hydrogen sulfide and ammonia. If a pure biomass were used as feedstock, this may not be needed owing to the fact that petrol gases used for the same purpose have had higher contact with sulfurous minerals in the earth: however, this system is designed also to handle materials with higher sulfur and ammonia content, such as garbage and sewage as well as the black liquor produced by paper mills. In addition, it is also prepared to handle biological gases and landfill gases where such materials toxic to catalysts are abundant. Those skilled in the art will notice that the system described needs a purge gas to remove inert gases from the system, such as nitrogen: thus, the purge gas is introduced at this point, both to negate the pressure drop associated with such scrubbing systems as well as to purify the incoming biological or landfill gas.

The outgoing gas from this system is then compressed adiabatically to heat the gas and prepare it for entry into the adiabatic pre-reformer. Research on hydrogen gasification systems and basic organic chemistry indicate that it is difficult to gasify or otherwise break apart aromatic compounds usually present as a result of gasification processes. Thus, such a system is needed to prevent coking of the steam reforming catalyst. A typical pre-reforming process is disclosed in U.S. Pat. No. 6,114,400, the entire disclosure of which is incorporated herein by reference for all purposes. It is recommended that a catalyst such as the Haldor-Topsoe H55N1 catalyst be used, as it has the power to break apart aromatic compounds such as benzene, toluene, and xylene into carbon monoxide, hydrogen, and carbon dioxide. These gases are then routed directly to the main steam reformer, where the gases are converted with steam into a synthesis gas over a commercial nickel catalyst at 1000 degrees Celsius. This reactor, different from the first two, is comprised of a series of bent metal piping, Schedule 40, in a firebox. This firebox can either be electrical or heated by the same landfill or biological gases used as the purge gas. In either case, the gas is heated to 1000 degrees Celsius and reformed to produce a mixture of carbon monoxide and hydrogen of an approximate initial ratio of 2.0 hydrogen to carbon monoxide.

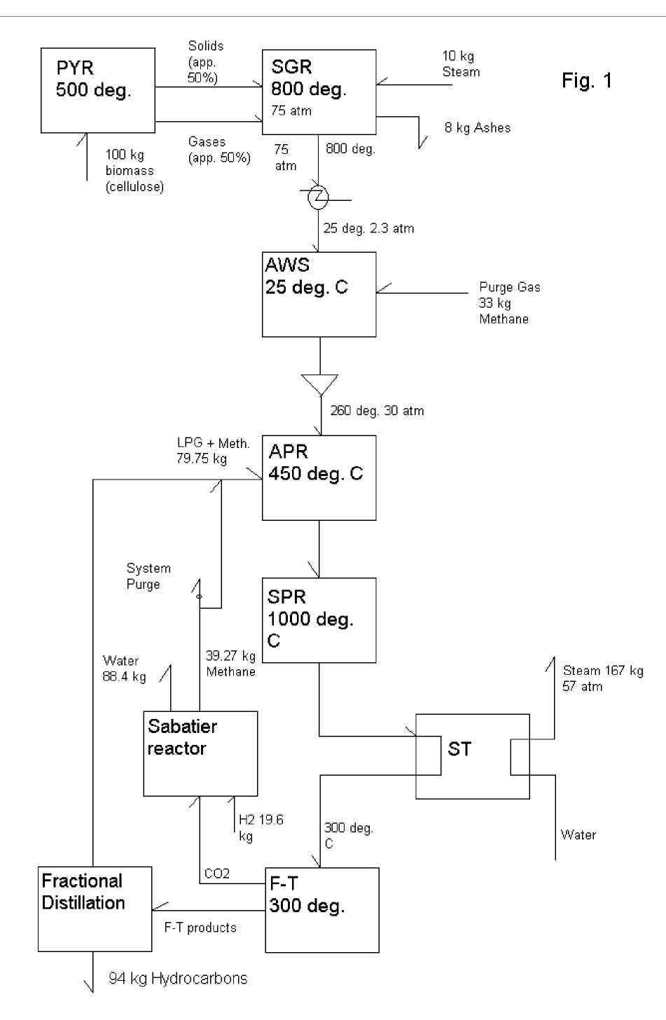

FIG. 1 shows a use for this synthesis gas for production of Fischer-Tropsch fuels. In this system, gases are first cooled by using the heat to create process steam, then F-T reaction conditions, including iron—cobalt catalyst chosen, are such that the process produces mainly gasoline (C5-C10) and LPG (C1-C4) fractions. The LPG fraction is fed back into the adiabatic pre-reformer, and the tail gas from the F-T process is sent through a Sabatier reactor over a nickel catalyst with 4 molar equivalents of hydrogen to form methane from any carbon dioxide in these gases. The hydrogen could be easily made during reforming of part of the gasoline fraction to aromatics to produce high-octane gasoline, though due to the presence of light hydrocarbons it is advisable to send this gas too through a pre-reformer. These gases are then fed back after removal of water into the pre-reformer for maximum carbon-to-carbon efficiency. The theoretical yield of this combined system is about 200 kilograms of F-T products, 94 kg hydrocarbons and 106 kg water. It is worth noting that this type of system can produce hydrocarbons suitable for lubricants and even waxes as well as motor fuels with modification of the Fischer-Tropsch reaction conditions, in a synthetic crude mixture that can be sold to refineries.

Also, those of skill in the art will know that such an enclosed loop system needs a purge gas to expel any buildup of inert gases such as nitrogen in the system. To this end, methane is added at the wash stage. This not only allows for this, but produces a higher yield of product and counters the pressure drop of such a system if the system is designed correctly. Adding it at the wash stage also allows the use of biological or landfill gases which may contain sizeable amounts of hydrogen sulfide and ammonia.

This synthesis gas can be also used to produce methanol via the ICI catalyst or to produce hydrogen via a water-gas shift. It is recommended that a secondary circuit be used with a water-gas shift reactor for the production of hydrogen for reduction, after carbon dioxide is removed.

While the present invention has been described with reference to specific embodiments, this application is intended to cover those various changes and substitutions that may be made by those of ordinary skill in the art without departing from the spirit or scope of the appended claims.

1. Fast pyrolysis of the carbonaceous material to bio-oil and char at 500 degrees Celsius.

2. Further reaction of the material in a circulating entrained flow gasifier with steam and/or oxygen, at a gas velocity of 2 to 10 meters per second and 1 to 25 atmospheres pressure.

3. Production of a synthesis gas from said reactor with no less than a 1.75 hydrogen/CO ratio, suitable for organic synthesis or power generation by burning the gas in a Brayton turbine.

4. Adiabatic recompression and adiabatic pre-reforming of the gases over a highly active nickel catalyst at 450 degrees Celsius.

5. Steam reforming of the gases at 1000 degrees Celsius over a commercial nickel catalyst for such purpose.

6. Production of a synthesis gas with a hydrogen to carbon monoxide ratio of approximately 2.0 suitable for production of methanol over the ICI copper/zinc oxide on alumina catalyst, Fischer-Tropsch fuel synthesis over an iron-cobalt catalyst, or hydrogen and carbon dioxide synthesis via a water-gas shift.
